(19) United States
(12) Patent Application Publication
(10) Pub. No.: US 2002/0111545 A1
(43) Pub. Date: Aug. 15, 2002

(54) Method and apparatus

(76) Inventors: Lars-Goran Lindberg, Linkoping (SE); Gunnar Enlund, Norrkoping (SE); Magnus Vegfors, Linkoping (SE)

Correspondence Address: BIRCH STEWART KOLASCH & BIRCH, PO BOX 747, FALLS CHURCH, VA, 22040-0747, US

(21) Appl. No.: 09/800,827

(22) Filed: Mar. 8, 2001

Related U.S. Application Data
(63) Continuation-in-part of application No. 09/657,306, filed on Sep. 7, 2000.

(51) Int. Cl. A61B 5/02

(52) U.S. Cl. 600/322; 600/504

(57) ABSTRACT

The present invention provides a non-invasive method for measuring blood circulation in tissue comprising the steps a) directing a first light beam and a second light beam coming from two different light sources with approximately the same wavelength against the tissue; b) detecting the intensity of the light of the first and second light beam, respectively, reflected from the tissue by using a detector appearing between said light sources; c) calculating a quotient of the detected intensity and a pre-determined zero-level intensity or a quotient of the detected intensity and a reference intensity; d) analyzing the quotient to determine the blood circulation; e) optionally, registering the quotient; and/or f) optionally, visualizing the quotient. The present invention also relates to and a computer program as well as an apparatus for performing said method.

### NEW METHOD AND APPARATUS

The present invention relates to a non-invasive method for measuring blood circulation in tissue comprising the steps, a) directing a first light beam and a second light beam coming from two different light sources with approximately the same wavelength against the tissue; b) detecting the intensity of the light of the first and second light beam, respectively, reflected from the tissue by using a detector appearing between said light sources; c) calculating a quotient of the detected intensity and a pre-determined zero-level intensity or a quotient of the detected intensity and a reference intensity; d) analysing the quotient to determine the blood circulation; e) optionally, registering the quotient; and/or f) optionally, visualising the quotient. The present invention also relates to a computer program as well as an apparatus for performing said method.

### BACKGROUND TO THE INVENTION

There is a method known in the art for identification of blood vessels, which is described in WO 98/44841, involving use of a motor system for positioning optical fibres for illuminating a vascular bed. One optical fibre is used for transmitting light and the other, which is connected to a detector, is used for detecting reflected light.

However, the above method lacks simplicity and is therefore not suitable for use during surgical operations.

Continuous temperature measurement is another method using one big toe as reference and the other big toe corresponding to the side of the body under operation. This method is however slow and not very accurate.

Another method for measuring circulation non-invasively is through using a laser doppler meter, see e.g. GB 2132483, which may be used only intermittently.

Another known method for measuring blood oxygen saturation non-invasively is disclosed through U.S. Pat. No. 5,007,423, where there are two light emitting diodes positioned with an optical detector in the middle in the same device. There is however no indications how to be able to determine blood circulation in deeper tissue.

Thus there is a need for a method and an apparatus for continuous, accurate and simple measurement of blood circulation.

### SUMMARY OF THE INVENTION

The present invention solves the above problems by providing according to a first aspect of the present invention a non-invasive method for measuring blood circulation in tissue comprising the steps

a) directing a first light beam and a second light beam coming from two different light sources with approximately the same wavelength against the tissue;

b) detecting the intensity of the light of the first and second light beams, respectively, reflected, from the tissue by using a detector appearing between said light sources;

c) calculating a quotient of the detected intensity and a pre-determined zero-level intensity or a quotient of the detected intensity and a reference intensity;

d) analysing the quotient to determine the blood circulation;

e) optionally, registering the quotient; and/or

f) optionally, visualising the quotient.

According to a second aspect of the present invention there is provided an apparatus for non-invasive measurement of blood circulation in tissue comprising:

i) at least two light sources which are capable of emitting light beams at approximately the same wavelength;

ii) at least one detector for detection of reflected intensity; and

iii) a processor for calculating a quotient of detected reflected intensity and a pre-determined zero-level intensity or a quotient of a detected reflected intensity and a reference intensity;

wherein the detector is appearing between the light sources.

Further, use of an apparatus according to the second aspect of the present invention for measuring blood flow in a flap is provided.

### DETAILED DESCRIPTION OF INVENTION

The term “light source” is to be understood to encompass in the present description one or more light emitting elements, such as one or more light emitting diodes (LEDs).

The expression “appearing between” is to be understood in the present description that a component appears precisely, on a straight line, between or essentially between two other components. The distance between the components may be varying as set out below.

The expression “haemoglobin” is meant in the present description to encompass total haemoglobin, oxyhaemoglobin, reduced haemoglobin, carboxy haemoglobin, methaemoglobin or sulphhaemoglobin.

As used herein, “light” refers generally to electromagnetic radiation at any wavelength, which includes the infrared, visible and ultraviolet portions of the spectrum. In this connection light of the portion of the spectrum, such as visible and near-infrared light, that at least partly is capable of penetrating tissue, is of particular interest. It should be understood that for the present invention, the light may comprise non-polarized or polarized light, coherent or incoherent light and illumination of the vessel may be carried out by using steady pulses of light, amplitude modulated light or continuous light.

The expression “is connected” is meant to embrace in the present description that components are linked together through a wire or through wireless transmission means by using e.g. a Bluetooth™ standard based communication path.

The expression “pre-determined zero-level intensity” is meant to embrace in the present description a level of intensity obtained when measuring circulation in a tissue, preferably a flap, to be transplanted to a new place of the body, which may be a human or an animal. This above intensity may be obtained long before or just before excising from the body, the affected tissue to be transplanted. This above intensity may be obtained by using the apparatus according to the present invention preferably fixed at the surface of the tissue before being operatively freed.

According to a further embodiment there is provided in the method according to the present invention that the light beams are directed essentially perpendicular against the tissue.

According to a further embodiment of the method according the present invention the light beams are having a wavelength of approximately 940 nm.

According to a further embodiment of the method according the present invention the light sources are positioned essentially in a row with the detector appearing between said light sources, preferably the distance between one light source and the detector is approximately 3 mm whereas at the same time preferably the distance between the other light source and the detector is approximately 10 mm. This positioning of the components above enables the measurement of blood circulation on a tissue depth deeper than 6 mm, which may preferably be approximately 12 mm. The distances given above, and which appears below in the rest of the present description, are distances taken from the centres of the respective components, if not other is indicated in a certain context.

According to a further embodiment of the method according the present invention the reference intensity is obtained by:

ra) directing a first reference light beam and a second reference light beam coming from two different reference light sources with approximately the same wavelength against a reference tissue;

rb) detecting the intensity of the light of the first and second reference light beam, respectively, reflected from the reference tissue by using a reference detector appearing between said reference light sources. Preferably the reference light beams are having a wavelength of approximately 940 nm. The reference tissue is preferably an untreated part of the body, most preferred a part of tissue appearing in a corresponding part of the body i.e. is essentially similar to the tissue which is to be or has been transplanted.

According to a further embodiment of the method according the present invention the reference light sources are positioned essentially in a row with the reference detector appearing between said reference light sources, preferably the distance between one reference light source and the reference detector is approximately 3 mm whereas at the same time preferably the distance between the other reference light source and the reference detector is approximately 10 mm.

According to a further embodiment of the method according the present invention the analysing of the quotient activates an alarm device when the quotient drops under a predetermined value which may e.g. be the value 0.9 or the value 90% or other values as set forth below. The means for visualisation, which may preferably be connected to the processor, may continuously display a figure which corresponds to the relative blood flow i.e. the quotient referred to above.

According to a further embodiment of the apparatus according the present invention the light sources emit light beams at a wavelength of approximately 940 nm.

According to a further embodiment of the apparatus according the present invention the light sources are positioned essentially in a row with the detector appearing between said light sources, preferably the distance between one light source and the detector is approximately 3 mm whereas at the same time preferably the distance between the other light source and the detector is approximately 10 mm, most preferred said light sources and detector are positioned in a straight line. The distance between the above components is essential for obtaining a suitable measuring depth which is further elaborated below. A measuring depth down to approximately 10 mm may be sufficient for detecting the flow on the venous and arterial side in the fixed tissue i.e. the flap of the locus for operation.

According to a further embodiment of the apparatus according the present invention the light sources and the detector are fixed in a patch device, preferably flexible and capable of being intimately fixed to the skin of a subject or a flap thereof.

According to a further embodiment of the apparatus according the present invention the patch device has bevelled edges, preferably rounded edges.

According to a further embodiment of the apparatus according the present invention the patch device is capable of being fixed to the skin of a subject or a flap thereof by using fixation means, preferably breathable adhesive tape or suturing wire. This tape may be Tegaderm which is a breathable, sterile, waterproof, transparent and light pervious tape (Tegaderm is a trademark). Thus this enables the fixation, with the help of an adhesive for greater staying power (accompanying Tegaderm), during more than 48 hours on the subject.

According to a further embodiment of the apparatus according the present invention an alarm device is connected to the processor.

According to a further embodiment of the apparatus according the present invention the apparatus further comprises:

iv) registration means for storing values of the determined blood circulation in tissue; and

v) optionally, means for visualisation of the determined blood circulation.

According to a further embodiment of the apparatus according the present invention the apparatus further comprises:

vi) at least two reference light sources which are capable of giving light beams at approximately the same wavelength;

vii) at least one reference detector for detection of reflected reference intensity;

preferably connected to said processor. Preferably the reference light sources emit light beams at a wavelength of approximately 940 nm. Thus an apparatus according to the invention may be fixed on a tissue, preferably a flap, whereas at the same time another essentially identical apparatus (reference apparatus; as set out above) is fixed on a reference tissue having a “normal” circulation. Preferably the reference tissue is appearing on a place of the body which corresponds to the tissue to be transplanted i.e. the flap. If e.g. a flap is excised from an arm, preferably the reference intensity is obtained by using a reference apparatus on the other arm. When the flap is in place the blood flow is monitored. It is essential that the venous and the arterial blood flow is adequate.

According to a further embodiment of the apparatus according the present invention the reference light sources are positioned essentially in a row with the reference detector appearing between said reference light sources, preferably the distance between one light source and the detector is approximately 3 mm whereas at the same time preferably the distance between the other light source and the detector is approximately 10 mm, most preferred said reference light sources and reference detector are positioned in a straight line.

According to a further embodiment of the apparatus according the present invention the reference light sources and the reference detector are fixed in a patch device, preferably flexible and capable of being intimately fixed to the skin of a subject. Preferably the patch device has bevelled edges, preferably rounded edges.

According to a further embodiment of the apparatus according the present invention the patch device is capable of being fixed to the skin of a subject or a flap thereof by using fixation means, preferably breathable adhesive tape or suturing wire.

According to a further embodiment of the apparatus according the present invention the apparatus further comprises means for non-invasive detection of haemoglobin. The means for non-invasive detection of haemoglobin may be devices and apparatuses disclosed in our co-pending applications PCT/SE00/01739, PCT/SE00/01740 and PCT/SE00/01741, all three hereby incorporated by reference thereto. Further there may be a parallel measurement of SpO₂ incorporated as set out in PCT/SE00/01740 above.

According to a further embodiment of the apparatus according the present invention the patch device is essentially quadratic and has approximate dimensions 10×20 mm.

According to a further embodiment of the apparatus according the present invention the patch device is essentially triangular.

According to a further embodiment of the present invention there is provided a computer program stored on a data carrier for performing steps (a), (b), (c), and optionally (e) and/or (f) of the method as set forth above. The method of working for the computer program particularly when using two apparatuses according to the present invention, one for the flap and the other for reference tissue, may involve one or more of the following steps:

1) when both of the apparatuses, preferably the patch devices, have been fixed onto their respective loci a calibration is performed;

2) the computer program tests whether there are two AC-signals with a sufficient quality;

3) the program senses the weighed median value of the signals during 30 seconds and locks the relative multiplying degree between the signals and shows this value as 100% (a Microindex as set out below);

4) the calibration may be repeated if necessary (this repeat may if necessary be abolished to prevent any human errors which could occur; thus not jeopardizing “the normal setting”);

5) registering of the measurement values every minute (on e.g. a micro chip) and optionally displaying on a means for visualisation (e.g. a computer display) the values graphically (optionally on a variable time scale from e.g. 1 to 48 hours);

6) when the value decreases below 90% an alarm device is activated, optionally indicating (preferably during flap surgery) an optimization of blood pressure and perfusion pressure to be started together with a fluid addition (future research may disclose other values at which an alarm device should be activated or when indications for start of treatment should be displayed);

7) when the value decreases below 80% an indication of starting infusion of Heparin or other thrombosis dissolving treatment may be displayed;

8) when the value decreases below 70% an indication of starting new surgery with unblocking of vessels or resuturation of blood vessels may be displayed;

9) if the value increases over 100% this may be displayed as a positive indication (if over 100% this shows that the blood flow in the monitored tissue is enhanced relatively);

10) the strength of the signals may be displayed in the flap patch device (in Lux or in mA) (This enables the assessment of the quality of the signal);

11) optionally displaying the pulse curve for the flap apparatus optionally together with the reference apparatus (This enables the clinicians to assess themselves if the basic signals are trustworthy, which affects the trust in the Microindex above or not).

The results of the calculation may be displayed as a weighed median value which may be updated each 15 to 30 seconds in the means for visualisation. The means for visualisation may incorporate Daquhura which enables the simultaneous display of Microindex (MI_{AC} or MI_{DC}) and at the same time registering the values e.g. in a spread sheet.

A preferred area for use of the method and the apparatus according to the present invention is within surgery, especially within micro-surgical flap technology, “free-flap”-transplantation where it is a critical that the blood circulation is sufficient within the flap, thus enabling for the flap to survive. The monitoring of the method and the apparatus according to the present invention may be used for assessing that venous and arterial blood flow is adequate. Sufficient venous and arterial blood flow is essential for the survival of the flap and a positive outcome of the operation. Other preferred areas for application of the method and the apparatus according to the present invention are:

all vascular surgeries

plastic surgeries, especially during flap surgeries

hand surgeries, when fingers or other parts of limbs needs to be sutured

trauma patients, when there is a suspicion of vascular damage

crush damages, where there is a suspicion of compartmental syndrome

during ongoing micro surgical and vascular operations.

Thus every operation unit and intensive care unit (ICU) may have use of the method and the apparatus according to the present invention.

Adequate blood flow is detected either by using one apparatus according to the present invention or by using two apparatuses (i.e. an additional reference apparatus) as set out above. When using the one apparatus approach the apparatus is calibrated through measurements on parts of the body comparable with the tissue to be transplanted i.e. the flap, having a normal circulation of the blood flow. A pre-determined zero-level intensity is thus obtained. Thereafter the apparatus is fixed on the flap and the blood flow is monitored therein. When using the two apparatuses approach, one of the apparatuses is fixed at the tissue to be transplanted or has been transplanted i.e. the flap, and the other apparatus is fixed on a part of the body comparable with the flap having a normal blood flow. From both of the apparatuses there are intensity signals coming in which may be: 940 AC and 940 DC. The quotient referred to above for the two apparatus approach may thus be stated:

MI=940 ACop/940 ACref

An alternative formula for the quotient may be

MI=(940 ACop/940 DCop)/(940 ACref/940 DCref)

wherein:

MI=microindex, which may be the figure displayed on the means for visualisation, optionally multiplied with 100 to obtain a percentage.

op=operation apparatus (fixed at the flap)

ref=reference apparatus (fixed at a reference tissue)

The formulas above may be used depending on the present situation.

A serious and common cause for complications after the transplantation is that the venous flow or the arterial flow is insufficient in the flap. In order to remedy a complication it is important to assess if it is a venous stasis or an arterial embolism. The PPG signal of e.g. an apparatus for non-invasive detection of haemoglobin enables separating between a venous stasis and arterial embolism. An apparatus for this is disclosed in our co-pending applications PCT/SE00/01739 and PCT/SE00/01741, both hereby incorporated by reference thereto. Further you may also measure SpO₂ simultaneously by including apparatuses disclosed in our co-pending applications PCT/SE00/01740, hereby incorporated by reference thereto. The above PPG-signal may be affected by both flow and measuring volume:

A venous stasis in the flap gives an increased amount of blood in the flap. The reflected AC signal will decrease moderately since the pulsations will appear less clearly. The reflected DC signal will decrease since the detected blood volume under the probe increases and more of the incident light is absorbed.

An arterial embolism gives a decreased blood volume in the flap. The reflected AC signal will decrease drastically and will come to an end. The reflected DC signal will increase since the detected blood volume under the probe decreases and less of the incident light is absorbed.

Communication means may be provided for wireless communication between various components of the apparatus, including the light sources, detector and processor, and optionally the registration means and visualising means. The communication means preferably comprises a separate module for transmitting and receiving signals, wherein the module is capable of sending and receiving signals by using a Bluetooth™ standard based communication path. The radio communication standard Bluetooth™ has opened the opportunity for cable-free equipment in the hospital environment. Bluetooth™ technology enables electronic devices to communicate with one another without cables. Bluetooth™ modules comprising a transmitter and receiver may replace cables in many applications. Bluetooth™ technology, developed by L M Ericsson, may use the ISM band 2.45 GHz and may ensure interruption-free communication. The system may work with quick frequency hopping of 1,600 hops per second. The output power from the transmitter may be low and may be adapted to work at a maximum distance of 10 meters. The distance between the wireless communicable components in the apparatus of the present invention may however be variable from 1 cm up to 10000000 miles.

The light sources may comprise one or more light emitting diodes, wherein the distance between each diode and the detector is from 3 to 12 mm, preferably 3 to 10 mm, when referring from the centres of the diode and detector. The light diodes may be incorporated in the same shell, e.g. a patch device, and be positioned on one common side of the measured object. The material in the patch device does preferably not contain allergenic substances and thus the patch device is preferably well tolerable to the skin of a mammal.

The vessels of the tissue having blood circulation which is to be monitored may be identified by proper choice of the separation between the light sources and the detector. The theoretical analysis and experimental verification of this optical technique has been presented by I. Fridolin, K. Hansson and L. -G. Lindberg in two papers which have been accepted and are to be published in Physics in Medicine and Biology (Optical non-invasive technique for vessel imaging I and II, Department of Biomedical Engineering, Link\overline{o}ping University, Sweden). The following is a summary of their analysis and experimental verifications.

Light reflection from human tissue depends on many parameters, such as optical wavelength, source-detector separation, size and aperture of the light source and detector and optical properties of the blood and tissues. The separation between the light source and the detector fibre was varied between five centre-to-centre distances: 2, 3, 4, 5 and 6 mm. The analysis agreed with the earlier conclusion that to increase the influence from deeper tissue on the measured signal, a larger light source-detector separation should be selected.

The resultant mathematical analysis and verified experimental results can be summarised as:

At larger separation values the photons forming maximum photon paths and detected by the photodetector originate from deeper layer than for short separation values. This is illustrated in FIG. 4. FIG. 4 is a schematic diagram of photon migration at two different source-detector separations and for different FL (α) (FL(0) and FL(π/2)). FL=fibre pair position relative the Lining of the vein. Two positions of the light source and the photodetector fibres relative to the lining of the vein were considered. An angle α is defined to characterise different positions. The abbreviation FL(0) means that the light source and the photodetector are positioned in parallel and FL(π/2) that the light source and the photodetector are positioned perpendicular to the vessel. Monte Carlo simulations have shown that for human tissues in the near infrared region photons penetrate approximately 2 mm before being detected if the separation is about 2 mm between the source and the detector.

Blood vessels in terms of veins may be determined at three vascular levels in combination with a fixed fibre diameter (1 mm) and according to;

a superficial vascular level (approximately 1 mm). This may be sufficient to set the minimal distance between the illuminating and detecting fibre (2 mm during the above experiments.

an intermediate vascular level (approximately 2 mm). The minimal distance between the illuminating and detecting fibre may preferably be 2-3 mm

a deep vascular level (approximately 3 mm). The distance between the illuminating and detecting fibre may preferably be greater than 3 mm.

The result of these referenced papers indicate that it is possible to determined blood circulation in a selected vascular bed of tissue comprising veins or arteries. If wrists (containing Radialis) or thicker parts of the body, like upper parts of the arms, are to be measured, when regarding blood circulation the above distances between the fibres (light sources and detector) may be from 6 to 12 mm. For thicker parts (like arms containing Brachialis) of the body the distance may be from 12 to 30 mm. When measuring on wrists or thicker parts of the body a pressure may preferably be put on the measurement locus. The method according to the present invention may further be used when measuring on vessels situated below the ankles (containing Dorsalis pedis). Thus the present invention may have light sources and detector on different distances as set out above depending on which measuring area is to be monitored, which enables reaching the aimed vessel and thus the detection of the blood circulation. The distance between detector and light source(s) may, as set out above, thus be from 1 to 20 mm depending on the measuring area.

The theoretical solution for light distribution in tissue, described in paper 2 of the above referenced papers, is the base for describing how blood circulation can be measured in reflection mode. Equation 32 in this paper provides a general solution in which equation μₐ and μₛ describes the influence of the optical coefficients and H and B (or Z) the influence on pulsatile variations in vessel diameter during the cardiac pulse.

The light sources are connected to any power source, which may be an oscillator. The oscillator may be connected to amplifiers and LED-Drivers. These drivers may be connected to one or more LEDs. Detectors, e.g. photodiodes, for reflection are connected to at least one current/voltage converter, which in turn may be connected to the amplifiers. The signals may then pass to Band pass Filters and subsequently to analog outputs or to a μ-controller which is connected to a Read out unit.

Suitably the processor is adapted to perform steps (a) to (d) of the new method. Furthermore, the processor may be adapted to convert the quotient of the detected intensity values to a percentage value of blood circulation. Generally, light sources for use in the method and apparatus of the invention may be light emitting diodes (LEDs) or laser diodes, such as vertical cavity surface emitting laser (VCEL). Preferably less expensive LEDs are used. Today there are also new strong light emitting diodes which may be used. Flash lamps, quartz halogen lamps or tungsten lamps may also be used as light sources. The light sources may further be capable of emitting monochromatic light, i.e. monochromators. The spot on the tissue to be measured may be directly illuminated or indirectly illuminated by guiding the light through optical fibres.

Detectors suitable for use in the method and apparatus of the invention, are phototransistors, photodiodes, photomultipliers, photocells, photodetectors, optical power meters, amplifiers, CCD arrays and the like.

The apparatus and the method according to the present invention has a high sensitivity. Further it is easily sutured on the flap or is fixed with surgical tape e.g. Tegaderm. Additionally it may be fixed during more than 48 hours. The patch device may be sterilized or be disposable i.e. made of an inexpensive material. The patch device allows for the fixation during ongoing operation, as micro surgery and vascular surgery may last from 4 to more than 24 hours.

We will now describe the present invention by using figures and an example but they are only for purposes of illustration and shall not in any way limit the scope of the appended set of claims.

### FIGURES

FIG. 1 shows a flap transplanted to the cheek with a patch device (triangular) according to the present invention sutured on the flap.

FIG. 2 shows a bad signal from the blood flow. The flap had to be resutured.

FIG. 3 shows a good signal from the blood flow.

FIG. 4 shows at larger separation values the photons forming maximum photon paths and detected by the detector originate from deeper layer than for short separation values.

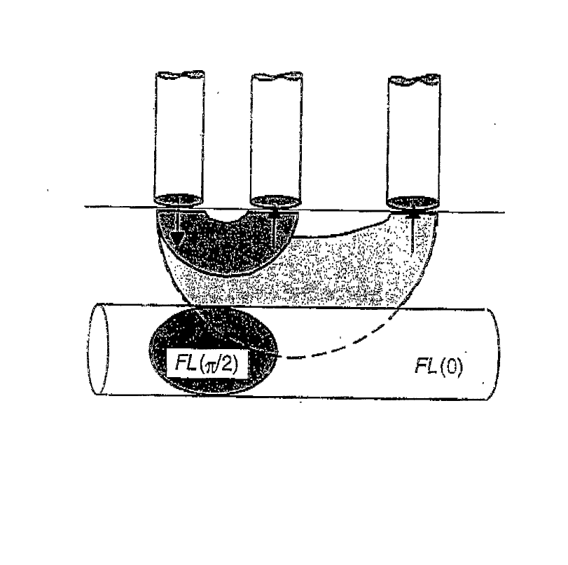

### EXAMPLE

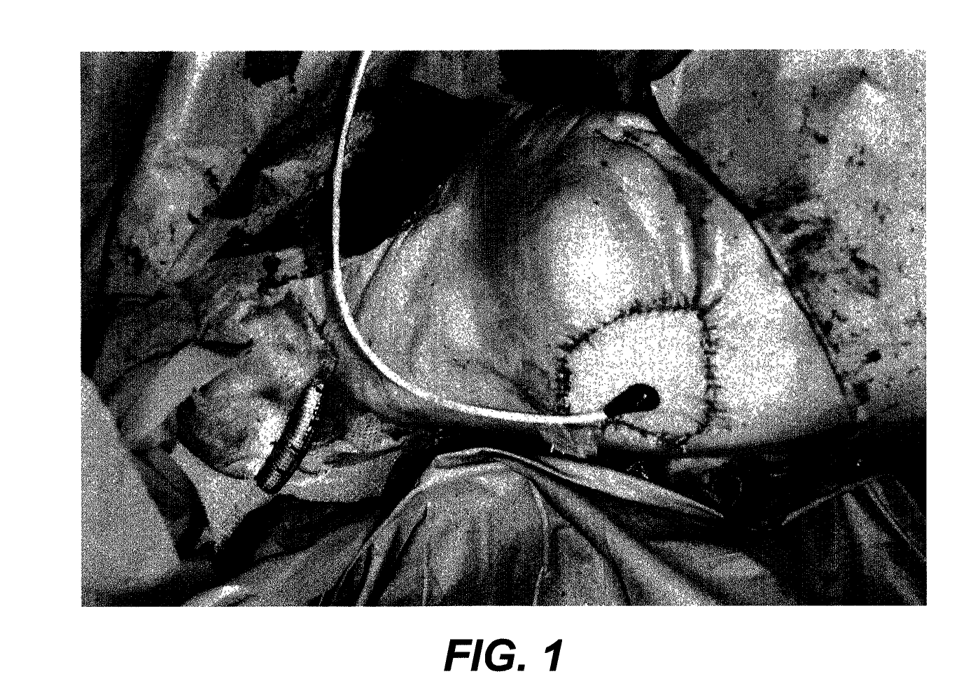

The patch device according to the present invention was used for monitoring blood circulation during operation of cancer whereby a free flap was transplanted on a subject. The operating theatre at the back had the flow meter, especially the visualisation means positioned. Cancer was taken away from the cheek. A free flap, to be transplanted into the cheek, was obtained from a healthy arm with a healthy tissue somewhat corresponding to the flap tissue. There was a vascular suture performed. FIG. 1 shows the flap transplanted to the cheek with a patch device according to the present invention sutured on the flap. The patch device is connected through a wire to a computer (the computer is not shown). The wire is appearing in the figure. You can also see the stitches appearing around the flap, which has been inserted into the cheek.

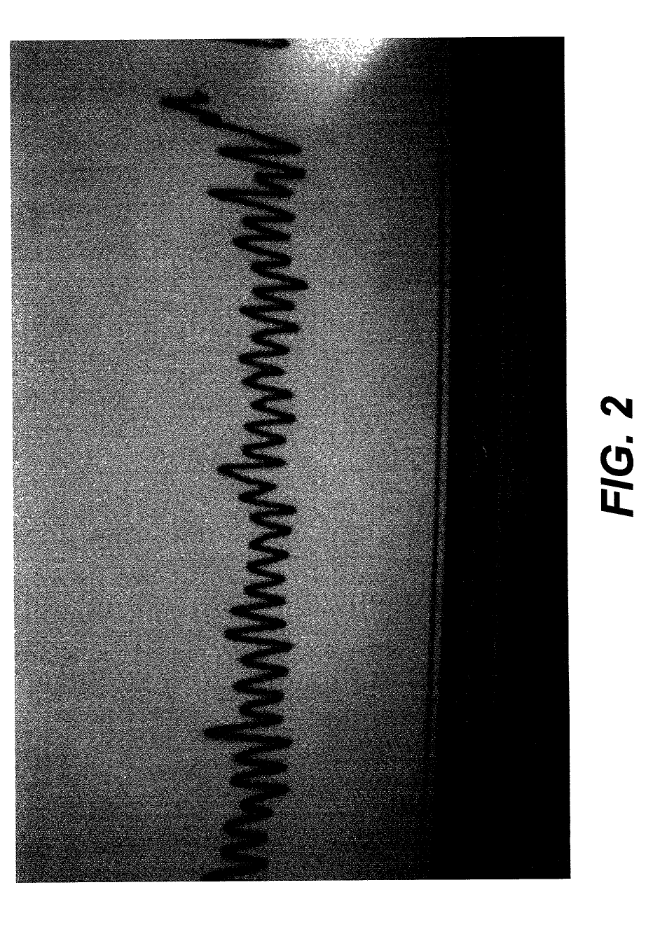

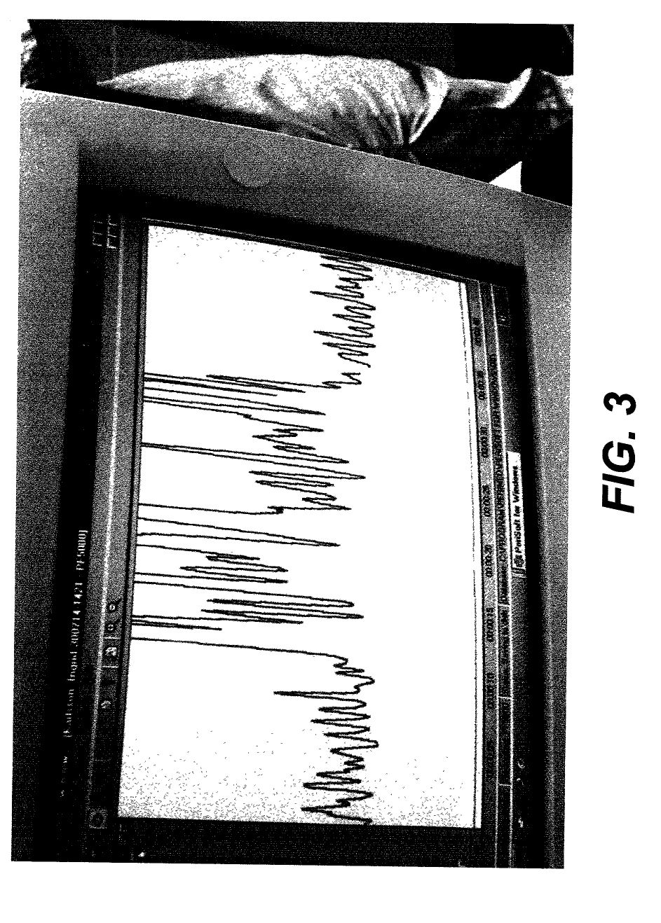

During the above free flap transplantation a visualisation means, i.e. a computer screen, was connected to the patch device thus enabling an easy overview of the circulation of the flap. FIG. 2 shows a signal from the blood flow which indicates that the flap has a bad circulation. The curve displayed on the computer screen has a low amplitude. The flap has to be resutured. FIG. 3 shows a signal from the blood flow which indicates a good circulation of the flap. The curve displayed on that computer screen of FIG. 3 has a high amplitude and shows a “viable pattern”. If a reference apparatus according to the present invention would have been used when transplanting the flap excised from an arm on the under side, a reference intensity may have been obtained by using a reference apparatus positioned on the other arm on a corresponding tissue.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1. A non-invasive method for measuring blood circulation in tissue comprising the steps
a) directing a first light beam and a second light beam coming from two different light sources with approximately the same wavelength against the tissue;
b) detecting the intensity of the light of the first and second light beam, respectively, reflected from the tissue by using a detector appearing between said light sources;
c) calculating a quotient of the detected intensity and a pre-determined zero-level intensity or a quotient of the detected intensity and a reference intensity;
d) analysing the quotient to determine the blood circulation;
e) optionally, registering the quotient; and/or
f) optionally, visualising the quotient.

2. A method according to claim 1 wherein the light beams are directed essentially perpendicular against the tissue.

3. A method according to claim 1 wherein the light beams are having a wavelength of approximately 940 nm.

4. A method according to claim 1 wherein the light sources are positioned essentially in a row with the detector appearing between said light sources, preferably the distance between one light source and the detector is approximately 3 mm whereas at the same time preferably the distance between the other light source and the detector is approximately 10 mm.

5. A method according to claim 1 wherein the reference intensity is obtained by:
ra) directing a first reference light beam and a second reference light beam coming from two different reference light sources with approximately the same wavelength against a reference tissue;
rb) detecting the intensity of the light of the first and second reference light beam, respectively, reflected from the reference tissue by using a reference detector appearing between said reference light sources.

6. A method according to claim 5 wherein the reference light beams are having a wavelength of approximately 940 nm.

7. A method according to claim 5 wherein the reference light sources are positioned essentially in a row with the reference detector appearing between said reference light sources, preferably the distance between one reference light source and the reference detector is approximately 3 mm whereas at the same time preferably the distance between the other reference light source and the reference detector is approximately 10 mm.

8. A method according to claim 1 wherein the analysing of the quotient activates an alarm device when the quotient drops under a predetermined value.

9. An apparatus for non-invasive measurement of blood circulation in tissue comprising:
i) at least two light sources which are capable of emitting light beams at approximately the same wavelength;
ii) at least one detector for detection of reflected intensity; and
iii) a processor for calculating a quotient of detected reflected intensity and a pre-determined zero-level intensity or a quotient of a detected reflected intensity and a reference intensity;
wherein the detector is appearing between the light sources.

10. An apparatus according to claim 9 characterised by that the light sources emit light beams at a wavelength of approximately 940 nm.

11. An apparatus according to claim 9 characterised by that the light sources are positioned essentially in a row with the detector appearing between said light sources, preferably the distance between one light source and the detector is approximately 3 mm whereas at the same time preferably the distance between the other light source and the detector is approximately 10 mm, most preferred said light sources and detector are positioned in a straight line.

12. An apparatus according to claim 9 characterised by that the light sources and the detector are fixed in a patch device, preferably flexible and capable of being intimately fixed to the skin of a subject or a flap thereof.

13. An apparatus according to claim 12 characterised by that the patch has bevelled edges, preferably rounded edges.

14. An apparatus according to claim 12 characterised by that the patch is capable of being fixed to the skin of a subject or a flap thereof by using fixation means, preferably breathable adhesive tape or suturing wire.

15. An apparatus according to claim 9 characterised by that an alarm device is connected to the processor.

16. An apparatus according to claim 9 further comprising:
iv) registration means for storing values of the determined blood circulation in tissue; and
v) optionally, means for visualisation of the determined blood circulation.

17. An apparatus according to claim 9 further comprising:
vi) at least two reference light sources which are capable of giving light beams at approximately the same wavelength;
vii) at least one reference detector for detection of reflected reference intensity;
preferably connected to said processor.

18. An apparatus according to claim 17 characterised by that the reference light sources emit light beams at a wavelength of approximately 940 nm.

19. An apparatus according to claim 17 characterised by that the reference light sources are positioned essentially in a row with the reference detector appearing between said reference light sources, preferably the distance between one light source and the detector is approximately 3 mm whereas at the same time preferably the distance between the other light source and the detector is approximately 10 mm, most preferred said reference light sources and reference detector are positioned in a straight line.

20. An apparatus according to claim 17 characterised by that the reference light sources and the reference detector are fixed in a patch device, preferably flexible and capable of being intimately fixed to the skin of a subject.

21. An apparatus according to claim 17 characterised by that the patch has bevelled edges, preferably rounded edges.

22. An apparatus according to claim 17 characterised by that the patch is capable of being fixed to the skin of a subject or a flap thereof by using fixation means, preferably breathable adhesive tape or suturing wire.

23. An apparatus according to claim 9 further comprising means for non-invasive detection of haemoglobin.

24. An apparatus according to claim 12 or 20, wherein the patch device is essentially quadratic and has approximate dimensions 10×20 mm.

25. An apparatus according to claim 12 or 20, wherein the patch device is essentially triangular.

26. A computer program stored on a data carrier for performing steps (a), (b), (c), and optionally (e) and/or (f) of the method according to claim 1.

27. Use of an apparatus according to any one of claims 9 to 25 for measuring blood flow in a flap.
